(19) United States
(12) Patent Application Publication
(10) Pub. No.: US 2024/0366818 A1
(43) Pub. Date: Nov. 7, 2024

(54) APPARATUS AND METHOD FOR INACTIVATING VIRUSES AND PATHOGENS IN HUMAN PLASMA UNITS

(71) Applicant: Trevor Percival Castor, Arlington, MA (US)

(72) Inventor: Trevor Percival Castor, Arlington, MA (US)

(73) Assignee: Aphios Corporation, Woburn, MA (US)

(21) Appl. No.: 18/031,603

(22) PCT Filed: Oct. 12, 2021
(86) PCT No.: PCT/US2021/054626

§ 371 Date: Apr. 12, 2023

(51) Int. Cl. A61L 2/00

(52) U.S. Cl. CPC A61L 2/0023; A61L 2202/14; A61L 2202/15; A61L 2202/181; A61L 2202/22

(57) ABSTRACT

The present invention is a physical pathogen reduction method and apparatus for controlling or eliminating transfusion-transmittable infections. This purely physical technique does not involve the use of heat, chemicals and/or irradiation, each of which has significant drawbacks in the pathogen reduction of human plasma. The invention inactivates both nonenveloped and enveloped viruses as well as pathogenic bacteria and parasites in units of human plasma, while retaining the natural biological activity. integrity and potency of the treated plasma. The method uses critical, near-critical or supercritical fluids for viral and pathogen reduction of units of donor blood plasma, using novel blood plasma bags. The apparatus is in the form of a bench-top or mobile transportable unit, which can be used in hospitals, blood banks, medical facilities and hot zones in developing countries for the clearance of viruses from human plasma.

### REFERENCES TO OTHER PATENTS

This application discloses a number of improvements and enhancements to the viral inactivation method and apparatus disclosed in U.S. Pat. No. 5,877,005 to Castor et al., which is hereby incorporated by reference in its entirety.

This application discloses a number of improvements and enhancements to viral inactivation method and apparatus disclosed in U.S. Pat. No. 6,465,168 to Castor et al., which is hereby incorporated by reference in its entirety.

This application discloses a number of improvements and enhancements to the method for inactivating viruses for use in vaccines as disclosed in U.S. Pat. No. 7,033,813 to Castor et al., which is hereby incorporated by reference in its entirety.

This application discloses a number of improvements and enhancements to the method for inactivating viruses as disclosed in published U.S. Patent Application No. 2006/0269928 to Castor, which is hereby incorporated by reference in its entirety.

This application is being filed simultaneously on the same date with related inventions as disclosed in U.S. Provisional Patent Applications Nos. 63/090,701, 63/090,711 and 63/090,713 to Castor, which are hereby incorporated by reference in their entirety.

### FEDERAL SUPPORT

Research leading to this invention was in part funded with government support awarded by National Heart, Lung and Blood Institute, NIH, DHHS, United States of America.

### FIELD OF INVENTION

The present invention is directed to methods and apparatus for inactivating enveloped and non-enveloped viruses and other pathogens in units of whole plasma to prevent transfusion-transmitted infections. The process and apparatus feature critical, supercritical, or near critical fluids for inactivation of viruses and pathogens. The apparatus may be a portable unit designed to operate at point-of-care use such as a blood bank or hospital.

### BACKGROUND OF THE INVENTION

Viruses of many types pose an increasing and serious worldwide threat to humans and animals. The recent emergence of SARS-COV-2, the etiologic agent of our ongoing COVID-19 pandemic that has taken almost 5 million lives around the world and over 700,000 in the United States, has caused catastrophic damage to human health and safety, and untold economic damage to both the developed but especially the developing world. The rapid spread of the Zika virus can have a significant impact on neurological disorders in unborn fetuses and potentially adults. Outbreaks of the extremely virulent Ebola virus, the periodic emergence of severe viral respiratory infections (e.g. SARS, MERS), recurrent outbreaks of potentially pandemic strains of influenza (e.g. H5N1), and the worldwide AIDS epidemic have highlighted the need for effective methods and devices for the inactivation and removal of pathogens from human blood plasma and plasma-derived products.

Emerging viruses like West Nile, the Mexican swine flu, and high-risk bioterrorism pathogens such as smallpox are all major threats to the safety of the human plasma supply chain. In addition to viruses, bacteria and parasites such as Babesia spp. and Plasmodium spp. can cause severe transfusion-transmitted infectious diseases. The causes of the more rapid emergence and spread of these “killer” viruses and pathogens are not entirely known, but they are thought to be caused by some combination of deforestation with urbanization of wild virus habitats, evolutionary mutations and rapid global travel.

Even with the increased emphasis on screening of donors and testing for pathogens, the risk of transmission of infection by blood products is currently estimated to be 1 in 205,000 for hepatitis B virus (HBV), 1 in 2 million for hepatitis C virus (HCV), and 1 in 2 million for HIV (NHLBI, NIH, 2016). These numbers are significantly higher when disaggregated by region or socio-demographic clusters within and outside of the US. In addition, viruses such as human T-cell lymphotropic viruses (HTLV) types I and II, human parvovirus B19, cytomegalovirus (CMV) and Epstein-Barr virus (EBV) pose potential risks following the transfusion of blood and blood components.

Viruses of major concern as pathogens in human blood plasma include such as human parvovirus B19 and the hepatitis A, B and C viruses (non-enveloped viruses), and the enveloped viruses like human immunodeficiency viruses HIV-1 and HIV-2, and herpes viruses (CMV, EBV, HHV-6, HHV-7, HHV-8). For most of these viruses, the short period between initial infection and seroconversion presents a window of significant transfusion risk as routine clinical tests are not sufficiently robust to detect the virus (Ziemann, 2013). CMV seroprevalence, for example, may range from 40%-100% depending on locale, and establishes as a life-long latent infection with severe morbidity to patients (Pamphilon 1999).

Annually, an estimated 3.8 million Americans are transfused with 28.2 million blood components derived from 12.8 million units of blood donated by apparently healthy volunteers. A rigorous scrutiny of blood donors and screening of donated blood for various serological markers have significantly reduced the mortality and morbidity due to transfusion-transmitted infectious diseases; however, some enzyme immunoassays used for routine screening may detect viral antigens or antibodies, but not the infectious agents themselves.

Moreover, reliance on patient disclosure and risk assessment questionnaires has previously failed to correctly identify virus infected blood donors (CDC, 2010). Thus, there could be an asymptomatic window period of infectivity responsible for a residual risk of post-transfusion infection. Whereas conservative data estimates 1 in every 1.5 million are at a risk of transfusion transmitted infection (TTI) in the U.S (CDC, 2010), increasing globalization of public health increases the risk of TTI to Americans substantially.

A number of emerging viruses such as SARS Coronavirus, Zika, West Nile, the Mexican swine flu, and other potential bioterrorism pathogens like smallpox are not conventionally screened for, but are of concern to the safety of the human plasma supply chain. Additionally, microorganisms like bacteria, Babesia spp (Becker et al., 2009) and Lyme, and parasites like malaria are also not conventionally screened for, but are a major threat of spreading diseases through transfusions (American Cancer Society, 2013).

Moreover, the high frequency episodes of emergent and seasonal infectious diseases like Ebola, Zika, MERS, SARS, West Nile virus (WNV), and rare yet fatal blood borne infectious agents like Prion Diseases and Variant Creutzfeldt-Jakob Disease (vCJD), continue to cause localized and global-level public health risk from transfusion transmitted infection (TTI). Combined, these risks supersede the risks reported within individual country borders (CDC, 2013). In many developing countries, the risk of being transfused with donor blood infected with at least one pathogen (including HIV, HCV & HBV) is substantially high, ranging from 9.5%-21.1% in Ethiopia (Tessema 2010, Noubiap 2013) to 1.2%-15% in China (Zhao-Hua, 2012; Mollah, 2004). These risks and those of emergent infectious epidemics like Zika, follow regional and socio-demographic trends, from which developed countries cannot be precluded.

New products and processes that address the variable but important global burden of TTI will provide safer blood products that will benefit public health and also provide significant commercial innovations.

Several pathogen inactivation technologies have been used, but they have drawbacks. Current approaches such as pasteurization, solvent-detergent (SD), UV irradiation, and chemical and photochemical inactivation are not always effective against a wide spectrum of pathogens. Known approaches are sometimes encumbered by process-specific deficiencies, and often result in denaturation of the biologics that they are designed to render safe. There are limited commercially available, FDA-approved technologies for the inactivation of non-enveloped viruses, which could pose a significant future threat to the safety of human plasma and biologics. Currently, there are no drugs effective against multiple viral agents.

In addition, the implementation of known approaches often requires complex apparatus and processes, which are not easily transportable, and thus cannot be used locally in a host country under all conditions, where pathogenic inactivation technology would most effectively be used.

There is an immediate need for a technology to inactivate viruses and other pathogens in units of human plasma, particularly in developing countries and hot zones, while maintaining the biologic integrity of the plasma.

### SUMMARY OF THE INVENTION

The present invention is a generally applicable technology, based on physical principles, for the inactivation of both enveloped and non-enveloped viruses in units of human plasma with minimal reduction in biological integrity and potency. This technology reduces the risk of transfusion-mediated transmission of known as well as unknown pathogens and potential bioterrorism threats.

The drawbacks of prior known approaches are remedied by the present invention. In one aspect, the present invention is a physical pathogen inactivation technology and apparatus, in the form of a bench-top unit, for the inactivation of both non-enveloped and enveloped viruses as well as pathogenic bacteria and parasites in units of human plasma. The bench-top unit is portable and easily transportable to where it is needed most, in hospitals, blood banks, and medical facilities in the United States and in host countries. No dedicated facilities are needed to practice the present invention.

In another aspect of this invention, this technology utilizes supercritical and near-critical fluids (SuperFluids™ or SFS). SFS are normally gases at ambient conditions of temperature and pressure, which when compressed, exhibit enhanced thermodynamic properties of solvation, penetration, selection and expansion. These gases are used to permeate and inflate virus and pathogen particles. When the pressure in the SFS-saturated particles is released, the particles rupture at their weakest points as a result of rapid phase conversion and the forces of expansion, and become inactive or noninfectious.

The present inventor has demonstrated that the CFI™ (critical fluid inactivation) process inactivates both enveloped viruses such as MuLV, VSV, Sindbis, HIV (all completely inactivated), TGE, and BDVD, and the non-enveloped viruses Polio, Adeno, EMC (complete), Reo, and Parvo, while preserving biological activity of the CFI-treated product. The aspect of preserving the protein integrity and biological activity of the CFI-treated product is a major advantage over prior technologies and approaches.

Conventional approaches for pathogen inactivation in biologics are not always effective against a wide spectrum of human and animal viruses, and are sometimes encumbered by process-specific deficiencies, and often result in denaturation of the biologicals that they are designed to protect. CFI pathogen inactivation technology gives pathogens the “bends,” inactivating them without damaging proteins and enzymes in medically important transfusion fluids such as human plasma.

In research collaboration with the National Institute of Biological Standards and Control (NIBSC), London, England, the inventor demonstrated that CFI technology inactivated more than 4 logs of human Parvovirus B19 (one of the smallest and most resilient viruses) in human plasma in a two-stage CFI unit in less than 20 seconds. The inventor also demonstrated that SFS can disrupt and inactivate microorganisms such as E. coli, thick-walled prokaryotes such as Bacillus subtilis, and tough eukaryotes such as Saccharomyces cerevisiae at same SFS conditions for inactivating viruses. At the inventor's company, Aphios Corporation, scientists and engineers have defined operating conditions for achieving >6 logs of virus inactivation of prototypical enveloped and non-enveloped viruses in pooled human plasma in a 2-stage laminar flow CFI unit with retention of >80% of protein integrity.

The effect of treatment with different ratios of SFS CO₂ and N₂O on pH and coagulation factors in human plasma was evaluated in a two-stage CFI unit used for optimizing SFS composition. A plasma optimized SFS mixture consisting of N₂O:CO₂:97.5:2.5 inactivated 3.4 logs of the enveloped BVDV in pooled human plasma in the two-stage CFI unit at 208 bars and 37° C., and 4.1 logs of the non-enveloped adenovirus Type 2 in FBS at 208 bars and 40°° C. in a recycling, single-stage CFI unit.

Scaling this process down to units of blood was deemed to be critical, resulting in the present invention, which is designed for complete characterization of CFI-treated units of human plasma using customized blood bags in a prototype bench-top CFI unit. Results are compared to extant data for a continuous-flow laminar-flow CFI unit.

The present invention, based on CFI technology, is a purely physical technique that does not involve the use of heat, chemicals and/or irradiation, each of which has significant drawbacks in the viral inactivation of human plasma. As such, while CFI is capable of inactivating wide classes of viruses, bacteria and parasites, it has negligible negative impact on biological integrity and potency of the treated fluids. The development, testing, and validation of this process used continuous-flow laminar flow devices. The technology has now been scaled down for treating individual units of human blood plasma using a novel, portable bench-top CFI device using customized blood bags. The potential impact of a generally applicable, physical technology for inactivating viruses and emerging pathogens with high retention of biological activity is highly significant. CFI™ technology will also be vital for developing countries and hot zones for the clearance of viruses from human plasma.

These and other features, aspects and advantages of the present teachings will be better understood with reference to the following drawings, description, examples, and appended claims.

### BRIEF DESCRIPTION OF THE DRAWINGS

FIG. 1 shows before-and-after TEM (Transmission Electron Microscopy) photomicrographs of normal viral activity before CFI and after CFI disruption and inactivation of bacteriophage Φ-6 virus;

FIG. 2 shows before-and-after SEM (Scanning Electron Microscopy) photomicrographs of normal viral activity before CFI and after CFI disruption and inactivation of yeast (Saccharomyces cerevisiae);

FIG. 3 is a process flow diagram of the CFIU™ bench-top unit;

FIG. 4 illustrates the disposable plasma unit bag according to the present invention; and

FIG. 5 illustrates the disposable plasma unit and CFI product bags connections to the CFIU™ bench-top unit according to the present invention.

### DETAILED DESCRIPTION OF THE PREFERRED EMBODIMENTS

Viruses of all types pose an increasing serious worldwide threat. The worldwide pandemic caused by the SARS-COV-2 virus and its variants, the rapid spread of the Zika virus, which can have a significant impact on neurological disorders in unborn fetuses and potentially adults, the recent outbreak of the extremely virulent Ebola virus, periodic emergence of SARS, recurrent outbreaks of potentially pandemic strains of influenza such as H5N1, the continuing epidemic of MERS and the worldwide AIDS epidemic have highlighted a persistent concern in the health-care community—the need for effective pathogen inactivation and removal techniques for human blood plasma and plasma-derived products.

CFI™ (Critical Fluid Inactivation) utilizes supercritical and near-critical fluids (SuperFluids™ or SFS™). SuperFluids™ are normally gases which, when compressed, exhibit enhanced thermodynamic properties of solvation, penetration, selection and expansion. These gases are used to permeate and saturate virus and pathogen particles. The SFS-saturated particles then undergo decompression and, as a result of rapid phase conversion, viruses inflate and rupture at their weakest points.

The present inventor has demonstrated that the CFI™ (critical fluid inactivation) process inactivates both enveloped viruses such as MuLV, VSV, Sindbis, HIV (all completely inactivated), TGE, and BDVD, and the non-enveloped viruses Polio, Adeno, EMC (complete inactivation), Reo, and Parvo viruses, while preserving biological activity of the CFI-treated product. In research collaboration with the National Institute of Biological Standards and Control (NIBSC), London, England, the CFI process inactivated more than 4 logs of human Parvovirus B19 (one of the smallest and toughest viruses) in human plasma in a two-stage CFI™ unit in less than 20 seconds.

It has also been demonstrated that SFS can disrupt and inactivate microorganisms such as E. coli, thick-walled prokaryotes such as Bacillus subtilis and tough eukaryotes such as Saccharomyces cerevisiae at viral inactivation SFS conditions. CFI can be used with viral reduction methods such as nanofiltration as an orthogonal method of pathogen clearance, and is versatile for refinement to treat cellular blood.

This invention embodies the design and construction of a portable and comparably versatile bench-top CFI unit for pathogen reduction of single units of human plasma, using conventional and customized blood plasma bags.

The present invention is a physical pathogen inactivation technology, or Critical Fluid Inactivation (CFI™), for the inactivation of both non-enveloped and enveloped viruses as well as pathogenic bacteria and parasites in human plasma, plasma protein products and biologics. CFI™ technology is applicable to both pooled human plasma and units of plasma, the more globally significant focus of the current application.

Currently, there is no commercially available, FDA-approved technology for the inactivation of non-enveloped viruses in units of pooled human plasma and biologics, and only one approved method for units of plasma, which can inactivate some, but not all known non-enveloped viruses. This dearth of FDA-approved pathogen inactivation technologies poses a significant future threat for known and new viruses in human plasma and biologics.

A number of approaches have been employed for the inactivation or removal of viruses in human plasma, harnessing therapeutic proteins derived from human plasma and preparation of recombinant biologics. These include heating or pasteurization; solvent-detergent technique; Ultraviolet (UV) irradiation; chemical inactivation utilizing hydrolyzable compounds such as β-propriolactone and ozone; and photochemical decontamination using synthetic psoralens. The major problems with pasteurization include long pasteurization times, deactivation of plasma proteins and biologics, and the use of high concentrations of stabilizers that must be removed before therapeutic use. The solvent-detergent (SD) technique is quite effective against lipid-coated or enveloped viruses such as HIV, HBV and HCV, but is ineffective against protein-encased or non-enveloped viruses such as HAV, EMC and parvovirus B19. The solvent-detergent technique is also burdened by the need to remove residual organic solvents and detergents before therapeutic use. The photochemical-psoralen method, while quite effective with a wide range of viruses, is burdened by potential residual toxicity of photoreactive dyes and other potentially carcinogenic or teratogenic compounds that must be removed after treatment.

However, the Cerus Intercept method that is effective against both enveloped and some but not all non-enveloped viruses has been recently approved by the FDA for the viral clearance of human plasma, red blood cells and platelets. HAV, HEV, B19, and Polio Virus are resistant to the Cerus inactivation process, but are sensitive to the present CFI technology. Moreover, the Intercept method is restricted to units of plasma and is not applicable to pools of plasma, an advantage that the CFI offers since it was initially developed for pools of human plasma. The major weakness of CFI is that it has not yet been optimized for cellular blood e.g. platelets, an advantage Cerus' Intercept offers. However, CFI offers superiority in breadth in the number, types and strains of pathogens completely inactivated, with an accompanying simplicity, versatility and cost-efficiency. Thus, current approaches are not always effective against a wide spectrum of human and animal viruses, are sometimes encumbered by process-specific deficiencies, and often result in denaturation of the target biologics.

CFI technology, which inactivates both enveloped and non-enveloped viruses, is applicable to both pooled human plasma and units of plasma. The potential impact of a generally applicable, physical technology for inactivating both enveloped and non-enveloped viruses and emerging pathogens with high retention of biological activity is thus very significant. Such a technology, especially when used with conventional pathogen reduction or removal methods such as nanofiltration, will help ensure a blood supply that is safe from emerging and unknown pathogens and bioterrorism threats. In addition to human plasma and human plasma proteins such as fibrinogen and immunoglobulins, the developed technology will also be applicable to monoclonal antibodies and transgenic molecules.

The technology could be very impactful in developed countries and in hot zones for both the rapid virus clearance of pooled human plasma and units of plasma. The inventor developed two prototypes of this technology with versatility and cost efficiency that include; (i) an inexpensive bench-top prototype device that uses customized blood bags and can be readily deployed at community-level points-of-need where outbreaks occur, and (ii) pilot and large-scale CFI units to maximize high throughput processing at blood banks and industries (Industrial prototype). Both prototypes operate under similar CFI process conditions and use similar principles for pathogen inactivation. The technology offers unique advantages not achievable by currently available competing products like that of SD and the Cerus Intercept.

CFI™ pathogen inactivation works, in part, by first permeating and inflating the virus particles with a selected Superfluid™ under pressure. The overfilled particles are then quickly decompressed, and the dense-phase fluid rapidly changes into gaseous state rupturing the virus particles at their weakest points—very much like the embolic disruption of the ear drums of a scuba diver who surfaces too rapidly. The disruption of viral structure and release of nucleic acids prevents replication and infectivity of the CFI treated viral particle.

SuperFluids™ (SFS) of interest are normally gases, such as carbon dioxide and nitrous oxide, at room temperature and pressure. When compressed, these gases become dense-phase fluids, which have enhanced thermodynamic properties of selection, solvation, penetration and expansion. The ultra-low interfacial tension of SuperFluids™ allows facile penetration into microporous structures. As such, SFS can readily penetrate and inflate viral particles. Upon decompression, because of rapid phase conversion, the overfilled particles are ruptured and inactivated (Castor et al., 1995, 1999, 2000, 2001, 2002, 2005, 2006).

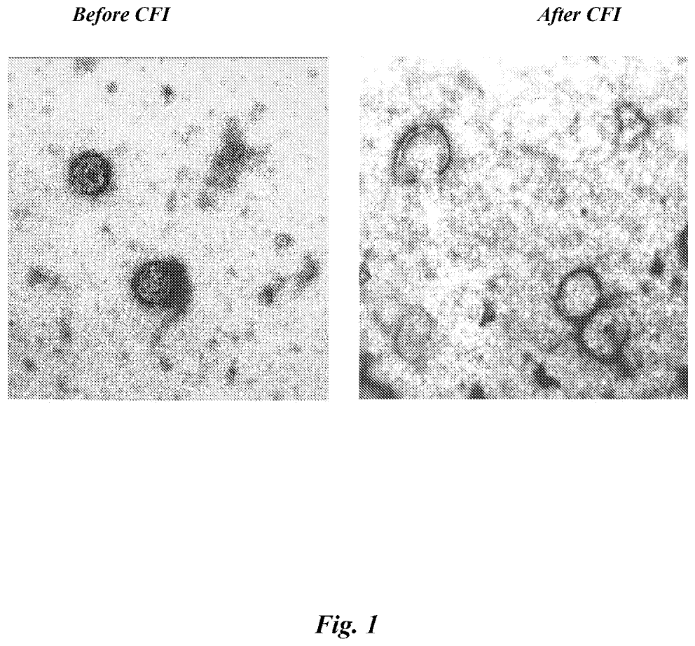

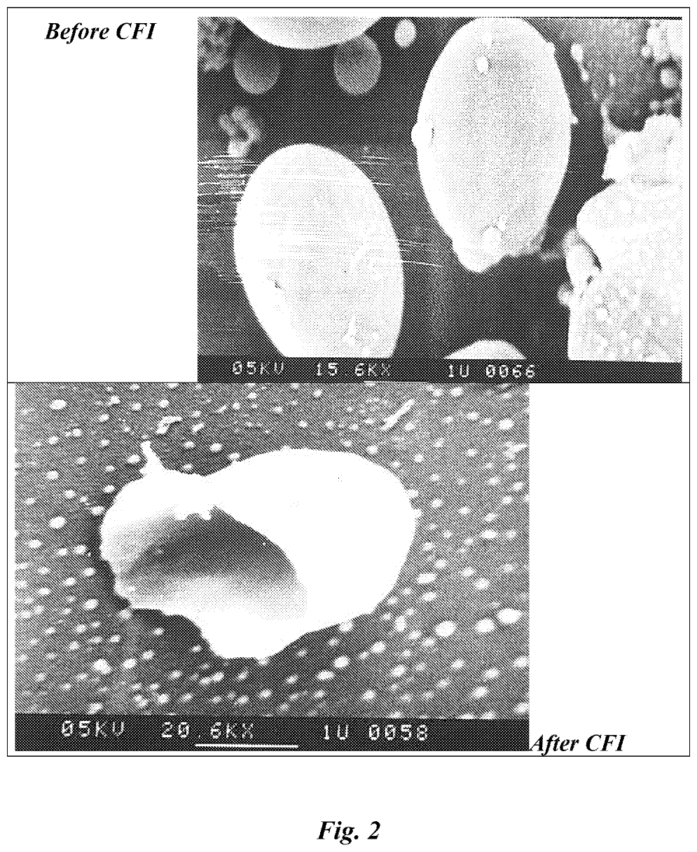

CFI has the capability to physically disrupt viral particles as shown by TEM stains of bacteriophage virus Φ-6 before and after CFI treatment in FIG. 1, and by SEM photomicrographs of yeast before and after CFI treatment in FIG. 2 illustrating its ability to inactivate enveloped viruses and a variety of other tough microorganisms. Also, like the SD technique developed by the New York Blood Center, CFI inactivates enveloped viruses by a lipid solubilization mechanism, dissolving away the protective lipid coat. The CFI process is compared to select commercially available virus inactivation processes in Table 1.

TABLE 1
Summary of Select Competitive Pathogen (Virus) Inactivation & Clearance Technologies

| Method/Company | Strengths | Weaknesses |
|---|---|---|
| CFI ™ | Effective against enveloped & non-enveloped | New technique requiring industry |
| Aphios | viruses | acceptance |
| Corporation | Applicable to units and pools of plasma | |
| | Near ambient temperatures; short processing | |
| | times | |
| | Gentle process conditions protect biological | |
| | activity | |
| | No removal of chemical additives required | |
| | Scalable with low operating costs | |
| Ultraviolet Light | Effective against enveloped and some non- | Process is not easily scalable |
| Activated Nucleic | enveloped viruses in platelet and plasma units | Not applicable to pools of plasma |
| Acid Modification | Able to be used at small blood processing | HAV, HEV, B19, and Polio Virus |
| Cerus Corporation | establishments | resistant to this inactivation process |
| | | Requires removal of a potentially |
| | | harmful chemical additive |
| Solvent/Detergent | Effective against enveloped viruses in pooled | Not directly effective against non- |
| Treated Pooled | plasma | enveloped viruses |
| Plasma | Able to be produced at a large scale | Requires removal of chemical additives |
| Octapharma | Widely accepted method | Loss of biological activity |
| | | Not applicable to units of human plasma |
| Nanofiltration | Effective against enveloped & non-enveloped | Passive process |
| Pall, Millipore, | viruses | Nonspecific removal of proteins |
| Asahi | Effective for smaller (<180,000 MW) proteins | Removes large proteins |
| | | Not applicable to units of human plasma |

Three fundamental steps are required for CFI pathogen clearance of protein-rich solutions containing viruses. SFS is first added to the product, which is then brought to the appropriate pressure and temperature conditions. Next, the aqueous sample is mixed with the SFS. Finally, the sample is decompressed to ambient pressure. The mixing step is an area of importance in the design and engineering of continuous flow CFI equipment, since most SFS and proteinaceous solutions are relatively immiscible with each other. Mixing will affect the efficiency with which virus particles are contacted and saturated with the SFS and their subsequent inactivation. Efficient mixing will also reduce processing time, improve manufacturing throughput and significantly reduce overall manufacturing costs.

Viral inactivation time can be significantly reduced and protein loss minimized by diffusing the SuperFluids™ into laminar, small-diameter aqueous droplets or streams. This discovery was made by modeling the mass transport phenomena that occurs between an SFS phase and a laminar flow protein-rich liquid phase. The inventor hypothesized that the disruption mechanism involved diffusion of the SFS from the suspending aqueous medium into the virus particle (virion) and vice-versa. If the pressure in the surrounding medium is reduced rapidly enough, fluids that had previously diffused into the virions do not have sufficient time to diffuse out again. The expansion of these fluids into gases within the virions will disrupt the viral structure. A model for this process would account for the diffusion of the SFS out of the virion in response to the time-varying boundary condition of SFS in the media surrounding the virus. This mechanism was modeled using Fick's Law of Diffusion through a series of spherical shells and solved the time-varying boundary condition for spherical coordinates by finite element analysis. Modeling of the explosive decompression mechanism gave guidance to operating pressures, pressure drop and rate of pressure drop.

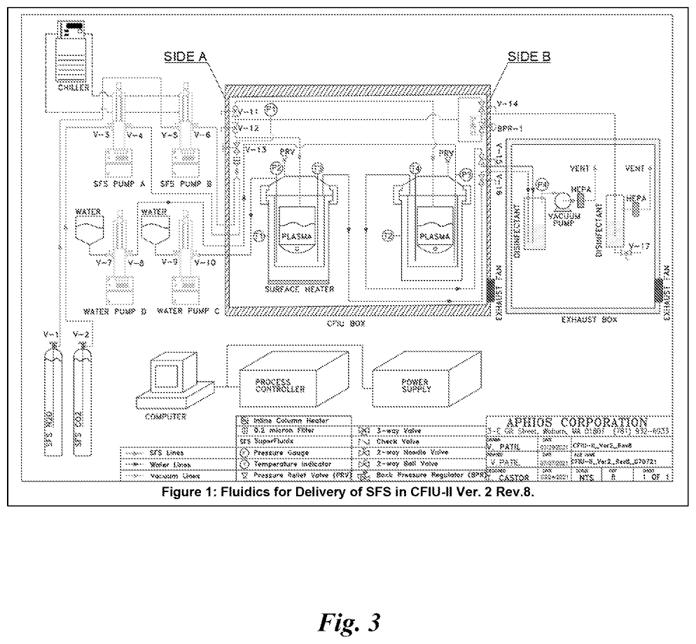

A bench top CFIU™ device design has been designed constructed and operated, and is shown in FIG. 3. High pressure fluidics for introduction of SFS in plasma bag are shown in FIG. 3. The system utilizes one (1) vacuum pump and four (4) syringe pumps which are operated using ISCO pump controller. The pressure and volume data from the pumps can be logged in real time using a laptop computer. A recirculating bath (VWR Model #1162) is used to cool the two (2) pumps used for delivering SFS to the system. PVC bags with capacity to hold 150 mL (Jorgenson Laboratories (Lot #20180926) are used as sample and CFIU processing or product bags. Two (2) 600 mL capacity pressure vessels, rated for a maximum allowable working pressure of 3,300 psig at 70° C. (Parr Instrument), are used in fluidics. The pressure vessels can be heated using a surface heater and controlled using a sensor, power supply and DC controller.

The process flow of the bench top CFIU™ is as follows: A CFIU bag containing plasma is introduced into pressure vessel #1 (PV-1) on Side B of the apparatus. Water or alternative hydraulic fluid is introduced into PV-1, by water pump D, external to the CFIU bag containing plasma, to maintain the vessel in an isobaric mode with ΔP<15 psig between the external water phase and plasma in the CFIU bag in PV-1. The temperature and pressure can be increased/adjusted to meet specified design conditions.

Liquid SFS is then proportionally introduced from SFS pump A (containing N₂O) and SFS pump B (containing CO₂) into the CFIU product bag in pressure vessel #2 (PV-2) on Side A of the apparatus by simultaneously decreasing the water volume/pressure in PV-2 with water pump C to maintain an isobaric mode with ΔP<15 psig between the external water phase and the SFS in the CFIU product bag in PV-2. The temperature and pressure can be increased/adjusted to meet specified design conditions.

The water pressure/volume in PV-1 is then increased at a pre-specified design rate to squeeze plasma from the plasma bag in PV-1 to the SFS product bag in PV-2 while simultaneously reducing the volume/pressure in PV-2 with water pump C to maintain an isobaric mode with ΔP<15 psig between the external water phase and the SFS in the CFIU product bag in PV-2. Once this transfer is completed, the product bag is isolated and decompressed in a single or multistep process. This CFIU process cycle is considered a single stage process.

The process using the CFIU cycle can be repeated by returning the plasma from the product bag using the hydraulics in the pressure vessels using water pumps C and D, and the process repeated for a 2-stage process, and again for a 3-stage process.

Alternatively, the process can be simplified by changing the functionality of the bags so that the sample bag becomes the product bag and vice-versa, diminishing the number of transfer steps by approximately 50% to achieve the same results. This change can be accomplished with a custom-designed plasma/product bag.

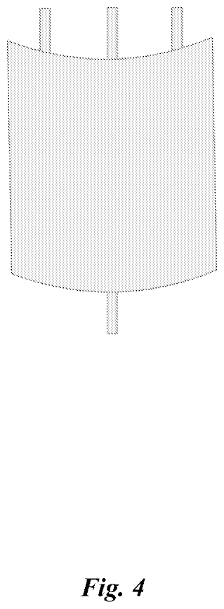

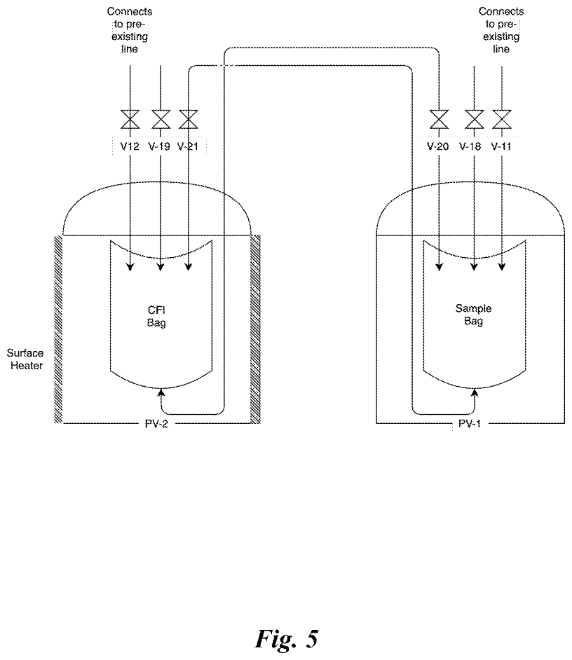

While the CFIU process can be operated with a standard 3-port on top plasma bag, operations can be simplified with a customized sample/product bag with three ports on top and one at the bottom as shown in FIG. 4. The customized blood plasma bag system consists of a single bag with four ports instead of the traditional three ports. Plasma is expressed into the bag through the middle port, which can then be heat sealed and separated from the original containment vessel. The two ports adjacent to the middle port host an outlet port and an injection port similar to those of a traditional blood component bag. One of the outermost ports hosts the SFS entry line, which consists of tubing with a disposable quick connector leading to an in-line disposable check valve. The other outer port hosts the SFS vent line, which consists of tubing with an in-line disposable vapor-liquid separator and/or demister, disposable sterile filter, check valve, and quick connector. The bottom port is used to transfer plasma from the sample bag to the CFI bag and from the CFI bag back to the sample bag. These ports can be positioned at various places on the bag including the bottom, the sides and the top as shown. These ports can be connected to fittings in the CFIU apparatus shown in FIG. 3 with some minor modifications. These modifications are shown in FIG. 5.

The customized plasma/product bag will be constructed of polytetrafluoroethylene (PTFE), commonly known by its brand name Teflon because of its compatibility with the utilized SFS 99:1 mixture of N₂O:CO₂. Other fluoropolymers such as perfluoroalkoxy alkanes (PFA) and fluorinated ethylene propylene (FEP) are alternative materials of construction given their similarities with PTFE. The customized plasma/product bag will consist of beat-sealed fluoropolymer (PFA, FEP or PTFE) bags with the required intubation.

For pathogen inactivation, bags are placed within pressure containment vessels with input lines connected to pre-existing lines as shown in FIG. 5. The vessel and the inside of the blood plasma bag systems are purged of air, then equally pressurized and warmed to a specified set point. The plasma is then transferred to the CFI bag containing SFS, and back again as many cycles or stages that are required. The input and output lines will then be heat sealed and the blood plasma bag systems, and the waste input and vent tube segments are removed from the system.

The bench-top device is highly versatile and will be useful in epidemic frontlines when safe blood products are an emergency need, are fairly affordable with low unit operating costs and no special technical skills are required for operation. Moreover, since the CFIU bags are disposable, there will be no requirement for between-operation sterilization.

The detailed description set forth above is provided to aid those skilled in the art in practicing the present invention. However, the invention described and claimed herein is not limited in scope by the specific embodiments herein disclosed. The embodiments are intended as illustrations of several aspects of the invention. Any equivalent embodiments are intended to be within the scope of this invention. Various modifications of the invention in addition to those shown and described herein will become apparent to those skilled in the art from the foregoing description which do not depart from the spirit or scope of the present inventive discovery. Such modifications are also intended to fall within the scope of the appended claims.

### EXAMPLES

### Example 1: Single-Stage CFIU™ Inactivation of Human Coronavirus-229E (HCV-229E) Virus Using 1% CO₂ in a SFS Mixture N₂O:CO₂:99:1 (CFIU-II-180)

In this single-stage CFIU-II-180 experiment, FBS was used instead of human plasma to avoid neutralization of human viruses by potential antibodies in donor plasma. The sample bag was loaded with 80 mL of SFS (N₂O:CO₂:99:1 at ˜2,250 psig and 40° C.) followed by ˜60 mL of FBS sample.

The virus titration results are listed in Table 2. The spike control showed a titer of 4.94 log TCID₅₀/mL, and the 4° C. control had a titer of 3.87 log TCID₅₀/mL, consistent with spiking the plasma with the virus stock at a 1:10 ratio. The Virus Reduction Factor (VRF) obtained for the CFI bag product before degassing was 1.61 log TCID₅₀.

TABLE 2
CFIU-II-180 - HCV-229E Virus Titration
Results (6 days Post Infection)

| CFIU-II-180 | Dilution | Number of | Titer (log | VRF (log |
|---|---|---|---|---|
| Sample | (3ⁿ) | wells+/8 | TCID₅₀/mL) | TCID₅₀) |
| 4° C. | 3 | 8 | 3.87 | 0.00 |
| Plasma | 4 | 7 | | |
| Control | 5 | 2 | | |
| | 8 | 1 | | |
| t&T | 3 | 8 | 3.87 | 0.00 |
| Plasma | 4 | 5 | | |
| Control | 5 | 2 | | |
| | 6 | 3 | | |
| CFIU Bag | 0 | 6 | 2.26 | 1.61 |
| Product | 1 | 8 | | |
| | 2 | 1 | | |
| | 3 | 0 | | |
| Spike | 5 | 8 | 4.94 | N/A |
| Control | 6 | 7 | | |
| | 7 | 4 | | |
| | 8 | 1 | | |

### Example 2: Single-Stage CFIU™ Inactivation of Encephalomyocarditis (EMC) Virus Using 1% CO₂ in a SFS Mixture N₂O:CO₂:99:1 (CFIU-II-178)

Several experiments were conducted with Encephalomyocarditis (EMC), a tough, prototypical non-enveloped or protein-encased virus to demonstrate the scalability of the CFI technology for non-enveloped viruses. EMC, a member of the Picornaviridae family, is a positive-strand RNA virus that is often used as a surrogate for the hepatitis A virus (HAV).

In the single stage CFIU-II-178 experiment, the sample bag was loaded with ˜80 mL of SFS (N₂O:CO₂:99:1 at ˜2,250 psig and 40° C.) followed by ˜58 mL of plasma sample. The virus titration results are listed in Table 3. The spike control showed a titer of 8.01 log TCID₅₀/mL, and the 4° C. control had a titer of 6.67 log TCID₅₀/mL. For the three control samples, there was no end point dilution with 0/8 wells. The Virus Reduction Factor (VRF) obtained for the CFI bag products before degassing was 2.98 log TCID₅₀.

TABLE 3
CFIU-II-178 EMC virus titration results (7 days post infection)

| CFIU-II-178 | Dilution | Number of | Titer | Titer (log | VRF (log |
|---|---|---|---|---|---|
| Sample | 3\hat{ }n) | wells+/8 | (3\hat{ }n) | TCID₅₀/mL) | TCID₅₀) |
| 4° C. | 9 | 8 | 10.63 | 6.67 | 0.00 |
| Plasma | 10 | 5 | | | |
| Control | 11 | 3 | | | |
| | 12 | 1 | | | |
| t&T | 8 | 8 | 10.13 | 6.43 | 0.24 |
| Plasma | 9 | 5 | | | |
| Control | 10 | 5 | | | |
| | 11 | 1 | | | |
| | 12 | 2 | | | |
| CFIU Bag | 2 | 8 | 4.38 | 3.69 | 2.98 |
| Product | 3 | 7 | | | |
| | 4 | 6 | | | |
| | 5 | 2 | | | |
| Spike | 11 | 8 | 13.44 | 8.01 | N/A |
| Control | 12 | 8 | | | |
| | 13 | 6 | | | |

The results of the FVIII coagulation assay of CFIU-II-178 are listed in Table 4.

TABLE 4
CFIU-II-178 - FVIII (Reference Range: 90 to 110% of the Control)

| CFIU-II-178 | Clot Time (Seconds) | | | Clot Time as % |
|---|---|---|---|---|
| Sample | Rep1 | Rep2 | Mean | 4° C. Control |
| 4° C. Control | 65.1 | 64.8 | 65.0 | 100% |
| t&T Control | 65.4 | 65.8 | 65.6 | 101% |
| CFIU Bag Product | 73.0 | 72.4 | 72.7 | 112% |
| CFIU Bag | 71.3 | 71.0 | 71.2 | 110% |
| Product - degassed | | | | |
| Fresh thaw | 59.6 | 58.7 | 59.2 | 91% |
| plasma Control | | | | |
| (Frozen - Apr. 5, 2021) | | | | |

The results of the SMAC analysis of CFIU-II-178 are listed in Table 5.

TABLE 5
SMAC Analysis for CFIU-II-178 Samples Before and After Degassing

| Test Name | Ref Range | | 178-4 | 178-T | 178-CP | 178-CPD |
|---|---|---|---|---|---|---|
| pH | 7.35-7.45 | | 7.49 | 7.51 | 7.23 | 7.42 |
| Albumin | 3.5-5.2 | g/dL | 3.6 | 3.5 | 3.6 | 3.7 |
| Bilirubin, Total | <1.2 | mg/dL | <0.2 | <0.2 | <0.2 | <0.2 |
| BUN (Blood | 6-23 | mg/dL | 8 | 6 | 7 | 8 |
| Urea Nitrogen) | | | | | | |
| Calcium | 8.6-10.4 | mg/dL | 7.3 | 7.3 | 7.4 | 7.4 |
| CO₂ | 19-29 | mmol/L | 16 | 15 | 17 | 15 |
| Chloride | 96-108 | mmol/L | 77 | 79 | 78 | 79 |
| Cholesterol | <200 | mg/dL | 113 | 112 | 114 | 116 |
| Creatinine | 0.67-1.31 | mg/dL | 0.75 | 0.78 | 0.81 | 0.84 |
| GGTP (gamma- | 10-71 | U/L | 14 | 14 | 14 | 14 |
| glutamyl | | | | | | |
| transpeptidase) | | | | | | |
| Iron | 59-158 | ug/dL | 58 | 57 | 68 | 67 |
| LD (Lactate | 135-225 | U/L | 121 | 123 | 120 | 121 |
| Dehydrogenase) | | | | | | |
| Phosphorus | 2.7-4.5 | mg/dL | 11.4 | 11.5 | 11.1 | 11.3 |
| Potassium | 3.5-5.5 | mmol/L | 3.6 | 3.6 | 3.6 | 3.7 |
| Total Protein | 5.9-8.4 | g/dL | 5.6 | 5.7 | 5.6 | 5.8 |
| AST (Aspartate | <40 | U/L | 15 | 18 | 17 | 18 |
| Aminotransferase) | | | | | | |
| ALT (Alanine | <41 | U/L | 6 | 6 | <5 | <5 |
| Aminotransferase) | | | | | | |
| Sodium | 135-147 | mmol/L | 165 | >165 | >165 | >165 |
| Triglycerides | <150 | mg/dL | 59 | 59 | 61 | 61 |
| Uric Acid | 3.4-8.5 | mg/dL | 3.5 | 3.5 | 3.5 | 3.5 |
| Alkaline | 40-156 | U/L | 53 | 54 | 55 | 56 |
| Phosphatase | | | | | | |
| A/G (Albumin/ | 1.1-2.9 | Ratio | 1.8 | 1.6 | 1.8 | 1.8 |
| Globulin) Ratio | | | | | | |
| BUN/Creatinine | 10.0-28.0 | Ratio | 10.7 | 7.7 | 8.6 | 9.5 |
| Ratio | | | | | | |
| Globulin | 1.7-3.7 | g/dL | 2.0 | 2.2 | 2.0 | 2.1 |
| Glucose | 70-99 | mg/dL | 644 | 628 | 630 | 635 |

The data listed in Table 3 indicate that 2.98 logs of inactivation were obtained for EMC in a single-stage CFIU unit. The FVIII clotting time was about 110% of the 4° C. control. In the SMAC analysis, the CFIU treated product (178-CP) and the degassed, treated product (178-CPD) showed negligible differences to the 4 C control (178-4) and the time and temperature control (178-T).

### Example 3: Two-Stage CFIU™ Inactivation of Encephalomyocarditis (EMC) Virus Using 1% CO₂ in a SFS Mixture N₂O:CO₂:99:1 (CFIU-II-185)

In the two-stage CFIU-II-185 experiment, the CFI bag was loaded with 80 mL of SFS (N₂O:CO₂:99:1 ˜2,250 psig and 40° C.) followed by ˜60 mL of plasma sample. The sample was transported back from CFI bag to Sample bag and then introduced into CFI bag carrying 80 mL of SFS in it.

The virus titration results are listed in Table 2. The spike control showed a titer of 7.56 log TCID₅₀/mL, and the 4° C. control had a titer of 6.85 log TCID₅₀/mL, somewhat consistent with spiking the plasma with the virus stock at a 1:10 ratio. The Virus Reduction Factor (VRF) obtained for the CFI bag product before degassing was 3.94 log TCID₅₀.

TABLE 6
CFIU-II-185 EMC virus titration results (7 days post infection)

| CFIU-II-185 | Dilution | Number of | Titer (log | VRF (log |
|---|---|---|---|---|
| Sample | (3ⁿ) | wells+/8 | TCID₅₀/mL) | TCID₅₀) |
| 4° C. | 10 | 5 | 6.85 | 0.00 |
| Plasma | 11 | 3 | | |
| Control | 12 | 3 | | |
| | 13 | 1 | | |
| t&T | 9 | 8 | 6.85 | 0.00 |
| Plasma | 10 | 7 | | |
| Control | 11 | 2 | | |
| | 12 | 2 | | |
| | 13 | 1 | | |
| CFIU | 1 | 8 | 2.91 | 3.94 |
| Bag | 2 | 4 | | |
| Product | 3 | 5 | | |
| | 4 | 1 | | |
| | 5 | 0 | | |
| Spike | 11 | 8 | 7.56 | N/A |
| Control | 12 | 4 | | |
| | 13 | 4 | | |
| | 14 | 0 | | |

The results of the FVIII coagulation assay of CFIU-II-185 are listed in Table 7.

TABLE 7
CFIU-II-185 - FVIII (Reference Range: 90 to 110% of the Control)

| CFIU-II-185 | Clot Time (Seconds) | | | Clot Time as % |
|---|---|---|---|---|
| Sample | Rep1 | Rep2 | Mean | 4° C. Control |
| 4° C. Control | 65.2 | 64.9 | 65.1 | 100% |
| t&T Control | 64.9 | 64.1 | 64.5 | 99% |
| CFIU Bag Product | 70.9 | 70.9 | 70.9 | 109% |
| CFIU Bag | 71.2 | 70.9 | 71.1 | 109% |
| Product - degassed | | | | |
| Fresh thaw plasma | 56.4 | 56.1 | 56.3 | 86% |
| Control (May 18, 2021) | | | | |

The results of the SMAC analysis of CFIU-II-185 are listed in Table 8.

TABLE 8
SMAC Analysis For CFIU-II-185 Samples Before and After Degassing

| Test Name | Ref Range | | 185-4 | 185-T | 185-80 | 185-CP | 185-CPD |
|---|---|---|---|---|---|---|---|
| pH | 7.35-7.45 | | 7.55 | 7.54 | ND | 7.37 | 7.58 |
| Albumin | 3.5-5.2 | g/dL | 3.3 | 3.2 | 3.8 | 3.2 | 3.3 |
| Bilirubin, Total | <1.2 | mg/dL | <0.2 | <0.2 | 0.2 | <0.2 | <0.2 |
| BUN (Blood Urea | 6-23 | mg/dL | 11 | 14 | 14 | 14 | 11 |
| Nitrogen) | | | | | | | |
| Calcium | 8.6-10.4 | mg/dL | 7.2 | 7.3 | 7 | 7.2 | 7.3 |
| CO2 | 19-29 | mmol/L | 14 | 14 | 15 | 15 | 13 |
| Chloride | 96-108 | mmol/L | 79 | 79 | 73 | 79 | 80 |
| Cholesterol | <200 | mg/dL | 165 | 162 | 173 | 164 | 165 |
| Creatinine | 0.67-1.31 | mg/dL | 0.8 | 0.72 | 0.81 | 0.76 | 0.85 |
| GGTP (gamma-glutamyl | 10-71 | U/L | 13 | 12 | 8 | 12 | 13 |
| transpeptidase) | | | | | | | |
| Iron | 59-158 | ug/dL | 106 | 107 | 125 | 122 | 119 |
| LD (Lactate | 135-225 | U/L | 123 | 122 | 103 | 122 | 122 |
| Dehydrogenase) | | | | | | | |
| Phosphorus | 2.7-4.5 | mg/dL | 10.5 | 10.5 | 11.2 | 10.8 | 10.6 |
| Potassium | 3.5-5.5 | mmol/L | 3.2 | 3.2 | 2.9 | 3.2 | 3.2 |
| Total Protein | 5.9-8.4 | g/dL | 5.2 | 5.1 | 5.7 | 5.2 | 5.3 |
| AST (Aspartate | <40 | U/L | 17 | 17 | 18 | 17 | 17 |
| Aminotransferase) | | | | | | | |
| ALT (Alanine | <41 | U/L | 12 | 9 | 12 | <5 | <5 |
| Aminotransferase) | | | | | | | |
| Sodium | 135-147 | mmol/L | 164 | 163 | >165 | 163 | 165 |
| Triglycerides | <150 | mg/dL | 103 | 102 | 119 | 104 | 105 |
| Uric Acid | 3.4-8.5 | mg/dL | 3.2 | 3.3 | 3.4 | 3.3 | 3.2 |
| Alkaline Phosphatase | 40-156 | U/L | 27 | 27 | 32 | 31 | 31 |
| A/G (Albumin/Globulin) | 1.1-2.9 Ratio | | 1.7 | 1.7 | 2 | 1.6 | 1.7 |
| Ratio | | | | | | | |
| BUN/Creatinine Ratio | 10.0-28.0 Ratio | | 13.8 | 19.4 | 17.3 | 18.4 | 12.9 |
| Globulin | 1.7-3.7 | g/dL | 1.9 | 1.9 | 1.9 | 2 | 2 |
| Glucose | 70-99 | mg/dL | 625 | 623 | 679 | 634 | 628 |

The data listed in Table 6 indicate that 3.94 logs of inactivation were obtained for EMC in a two-stage CFIU unit, an increase of about 1 log. The FVIII clotting time was about 109% of the 4° C. control, consistent with the single-stage results. In the SMAC analysis, the CFIU treated product (185-CP) and the degassed, treated product (185-CPD) showed negligible differences to the 4° C. control (185-4), the time and temperature control (185-T) and the −80° C. control (185-80).

### Example 4: Three-Stage CFIU™ Inactivation of Encephalomyocarditis (EMC) Virus Using 1% CO₂ in a SFS Mixture N₂O:CO₂:99:1 (CFIU-II-189)

In the three-stage CFIU-II-189 experiment, the CFIU bag was loaded with 80 mL of SFS (N₂O:CO₂:99:1 at ˜2,250 psig and 40° C.) followed by ˜60 mL of plasma sample. Twice, the sample was transported back from CFI bag to Sample bag and then introduced into CFI bag carrying 80 mL of SFS in it. The virus titration results are listed in Table 9. The spike control showed a titer of 7.68 log TCID50/mL, and the 4° C. control had a titer of 6.79 log TCID₅₀/mL, somewhat consistent with spiking the plasma with the virus stock at a 1:10 ratio. The Virus Reduction Factor (VRF) obtained for the CFI bag product before degassing was 5.07 log TCID₅₀.

TABLE 9
CFIU-II-189 EMC virus titration results (7 days post infection)

| CFIU-II-185 | Dilution | Number of | Titer (log | VRF (log |
|---|---|---|---|---|
| Sample | (3ⁿ) | wells+/8 | TCID₅₀/mL) | TCID₅₀) |
| 4° C. | 9 | 8 | 6.79 | 0.00 |
| Plasma | 10 | 5 | | |
| Control | 11 | 4 | | |
| | 12 | 2 | | |
| t&T | 8 | 8 | 6.97 | −0.18 |
| Plasma | 9 | 8 | | |
| Control | 10 | 7 | | |
| | 11 | 4 | | |
| | 12 | 3 | | |
| CFIU | 0 | 5 | 1.72 | 5.07 |
| Bag | 1 | 1 | | |
| Product | 2 | 0 | | |
| | 3 | 0 | | |
| | 4 | 0 | | |
| Spike | 11 | 8 | 7.68 | N/A |
| Control | 12 | 7 | | |
| | 13 | 3 | | |
| | 14 | 0 | | |

The data listed in Table 6 indicate that 5.07 logs of inactivation were obtained for EMC in a three-stage CFIU unit, an increase of about 1 log over the two-stage experiments and about 2 logs over the single-stage experiment.

What is claimed is:

1. A method for the pathogen reduction of viruses and other pathogens in single units of plasma by transferring the plasma for the sample bag to a product bag containing SuperFluids at a specified pressure and temperature, and decompressing the product bag to separate the SuperFluids from the product.

2. The method of claim 1 where the blood plasma is in a bag having one or more ports.

3. The method of claim 2 wherein there are 3 ports on top of the bag and one port on the bottom of the bag.

4. The method of claim 2 wherein the bag is made of polyvinyl chloride (PVC), polytetrafluoroethylene (PTFE), perfluoroalkoxy alkanes (PFA) or fluorinated ethylene propylene (FEP).

5. The method of claim 4 wherein the bag is made of polytetrafluoroethylene (PTFE).

6. The method of claim 1 wherein the SuperFluids are nitrous oxide (N₂O) and carbon dioxide (CO₂).

7. The method of claim 6 wherein the ratio of N₂O to CO₂ ranges from 90% to 100% N₂O, and from 10% to 0% CO₂.

8. The method of claim 6 wherein the ratio of N₂O to CO₂ 99% N₂O to 1% CO₂

9. The method of claim 6 wherein the SuperFluids are at a pressure of 2,000 to 5,000 psig and a temperature of 20° C. to 50° C.

10. The method of claim 9 wherein the SuperFluids are at a pressure of 2,500 to 3,500 psig and a temperature of 35 to 40° C.

11. The method of claim 10 wherein the SuperFluids are at a pressure of 3,000 psig and a temperature of 37° C.

12. A method for the pathogen reduction of viruses and other pathogens in single units of plasma by transferring the plasma for the sample bag to a product bag containing SuperFluids at a specified pressure and temperature, transferring the plasma back to the sample bag and back to the product bag and decompressing the product bag to separate the SuperFluids from the product.

13. The method of claim 12 wherein the plasma is transferred n times between the sample bag and the product bag before decompressing the product bag to separate the SuperFluids from the product.

14. The method of claim 13 wherein n=3 to 10.

15. The method of claim 14 wherein n=3.

16. An apparatus for inactivating viruses and other pathogen in units of blood plasma, comprising:
(a) a pressure vessel containing plasma in a sample bag surrounded by a hydraulic fluid;
(b) a pump for increasing or decreasing the volume or pressure of the hydraulic fluids surrounding the sample bag;
(c) a pressure vessel containing SuperFluids in a product bag surrounded by a hydraulic fluid;
(d) a pump for increasing or decreasing the volume or pressure of the hydraulic fluids surrounding the product bag;
(e) a pump for introducing a SuperFluids into the product bag;
(f) a pump for introducing a second SuperFluids into the product bag;
(g) chillers for maintaining the SuperFluids in a liquid state;
(h) heaters for maintain the temperature of the hydraulic fluids in the pressure vessels;
(i) connecting lines to move fluids from the sample bag to the product bag;
(j) a back-pressure regulator to contain and release pressure in the apparatus; and
(k) controllers for managing volumes, pressures and temperatures.

17. The apparatus of claim 16 wherein the hydraulic fluid is oil or water.

18. The apparatus of claim 17 wherein the hydraulic fluid is water.

19. The apparatus of claim 16 wherein the sample and product bags are multiport plastic bags.

20. The apparatus of claim 19 wherein the multiport plastic bags are made of polyvinyl chloride (PVC), polytetrafluoroethylene (PTFE), perfluoroalkoxy alkanes (PFA) or fluorinated ethylene propylene (FEP).
